(19) United States
(12) Patent Application Publication
(10) Pub. No.: US 2023/0011489 A1
(43) Pub. Date: Jan. 12, 2023

(54) SYSTEM AND METHOD FOR DIAGNOSING PULSATILE TINNITUS AND OTHER BLOOD VESSEL DISORDERS

(71) Applicant: The Regents of the University of California, Oakland, CA (US)

(72) Inventors: Matthew Amans, San Francisco, CA (US); Wade Smith, San Francisco, CA (US); Van Halbach, San Francisco, CA (US)

(21) Appl. No.: 17/783,468

(22) PCT Filed: Dec. 8, 2020
(86) PCT No.: PCT/US2020/063742

§ 371 Date: Jun. 8, 2022

Related U.S. Application Data
(60) Provisional application No. 62/945,601, filed on Dec. 9, 2019.

(51) Int. Cl. A61B 5/12; A61B 7/02; A61B 5/00; A61B 5/02

(52) U.S. Cl. CPC A61B 5/128; A61B 7/023; A61B 5/6852; A61B 5/02007

(57) ABSTRACT

A catheter for diagnosing an abnormality of a blood vessel includes an elongated body defining an elongated lumen therethrough; and one or more transducers disposed on an outer surface of the elongated body. One or more transducers are configured to output an electrical signal in response to sound.

### CROSS-REFERENCE TO RELATED APPLICATION

This application claims the benefit of and priority to U.S. Patent Provisional Application No. 62/945,601, filed on Dec. 9, 2019. The entire disclosure of the foregoing application is incorporated by reference herein.

### BACKGROUND

Various blood vessel abnormalities result in vascular turbulence or pressure fluctuations in the wall of the blood vessel, which in turn, results in sound generation. Certain conditions, such as pulsatile tinnitus, are caused by irregular blood flow. Pulsatile tinnitus is rhythmic and accounts for about 10% of tinnitus patients. A common mechanism of sound generation is attributed to flow patterns in blood vessels near the cochlea, the sound sensing cavity of the inner ear. In particular, pulsatile tinnitus may be caused by abnormal pulse-synchronous blood flow in vascular structures disposed near the cochlea, such as, transverse sinus, sigmoid sinus and internal jugular vein (“SSIJ”). The vascular structures with abnormal flow may be either venous or arterial. Approximately 40% of pulsatile tinnitus etiologies are due to abnormal venous flow, approximately 35% are due to arterial abnormalities, with the remainder of the cases being unidentified.

Many patients with pulsatile tinnitus have a source of sound generation inside of their blood vessels that is not easy to identify. Thus, there is a need for systems and method to precisely localize the source of sound generation due to blood vessel abnormalities for diagnosing pulsatile tinnitus and other vascular disorders.

### SUMMARY

Pulsatile tinnitus is a debilitating symptom that may be caused by a variety of vascular abnormalities, thus making the diagnosis very difficult. There are an estimated 3-5 million Americans that suffer from pulsatile tinnitus. In addition, vascular turbulence, abnormal wall sheer stress, and pressure fluctuations have been implicated in the development and progression of many vascular diseases throughout the body. The most common of these vascular diseases is an atherosclerotic disease involving the internal carotid arteries of the neck, but also potentially involving the coronary arteries, peripheral arteries, and intracranial arteries. Vascular turbulence is often involved in pathogenesis of cerebrovascular diseases such as atherosclerosis, dissections, aneurysm development, dural arteriovenous fistula, arteriovenous malformations, and the like.

The present disclosure provides a catheter having one or more transducers configured to precisely localize a source of sound generation due to vascular turbulence or pressure fluctuations that is responsible for a variety of vascular diseases, including pulsatile tinnitus. The catheter would facilitate early and accurate diagnosis. In particular, identifying turbulent flow in the blood vessels may be used to predict development of disease and/or identify progression of a disease prior to anatomic changes by identifying the flow perturbation, which may lead to earlier and safer therapies.

The catheter includes an integrated circuit having a transducer disposed at a distal portion of the catheter. The transducer is configured to convert sound or other pressure wave aberrations generated by blood flow from within the blood vessels and generate electrical signals in response thereto. The integrated circuit may be secured using a waterproof sealant or other adhesives. The integrated circuit includes one or more wires coupled thereto. The wires are disposed within the catheter and electrically couple the integrated circuit to an interface device having a driver circuit that is disposed outside of the patient. The interface device provides for amplification, recording, and identification of a source of sound within the patient's blood vessels.

Conventional devices, such as ultrasound devices, utilize sound waves to identify changes in anatomy or flow velocity, but are incapable of detecting sound or other turbulence by transducing the sound of the flow in the blood vessel. These conventional technologies insonate the vessel or flow and only receive the echoes of the induced sound waves.

According to one embodiment of the present disclosure, a catheter for diagnosing an abnormality of a blood vessel is disclosed. The catheter includes: an elongated body defining an elongated lumen therethrough; and one or more transducers disposed on an outer surface of the elongated body. One or more transducers are configured to output an electrical signal in response to sound.

According to another embodiment of the present disclosure, a system for diagnosing an abnormality of a blood vessel is disclosed. The system includes a catheter having an elongated body defining an elongated lumen therethrough; and one or more transducers disposed on an outer or inner surface of the elongated body. One or more transducers are configured to output an electrical signal in response to sound. The system also includes an interface device coupled to the at least one transducer, the interface device including a controller configured to process the electrical signal and to record the sound.

According to one aspect of any of the above embodiments, the elongated body has an outer diameter from about 0.5 mm to about 3.0 mm.

According to another aspect of any of the above embodiments, the catheter includes a plurality of transducers. The transducers are arranged in an array, which may be a linear array or a spiral array.

According to a further aspect of any of the above embodiments, the catheter includes a battery disposed within the elongated lumen, the battery coupled to the at least one transducer.

According to one aspect of the above embodiment, the controller is further configured to output the electrical signal through an audio output.

According to another aspect of the above embodiment, the interface device further includes a memory device configured to store a record of the sound. The memory device is configured to store a database of a plurality of entries, each of which pertains to a record of the sound.

Each entry of the plurality of entries includes at least one property describing the sound, the at least one property selected from the group consisting of amplitude, frequency, rhythm, and synchronicity.

According to a further aspect of the above embodiment, the memory device stores a diagnosing tool executable by the controller, the diagnosing tool configured to automatically diagnose an abnormality of a blood vessel based on the plurality of entries of the database.

According to a further embodiment, a method for diagnosing an abnormality of a blood vessel is disclosed. The method includes placing a catheter into a blood vessel near the cochlea, the catheter including an elongated body defining an elongated lumen therethrough; and measuring a sound at a transducer disposed on an outer or inner surface of the elongated body, the transducer configured to output an electrical signal in response to sound. The method further includes processing the electrical signal at a controller to determine location of the sound.

According to one aspect of the above embodiment, the sound is measured while the catheter is being withdrawn from the blood vessel.

According to another aspect of the above embodiment, the method further includes comparing the sound at the controller to a database of a plurality of sounds and corresponding parameters to automatically diagnose the abnormality, wherein the abnormality is at least one of an atherosclerosis, a dissection, an aneurysm, a dural arteriovenous fistula, an arteriovenous malformation, or pulsatile tinnitus.

According to a further embodiment of the present disclosure, a method for diagnosing and treating pulsatile tinnitus. The method includes obtaining one or more images of a vessel including a malformation and generating a computer three-dimensional model of the vessel. The method further includes manufacturing an original physical three-dimensional model of the vessel and flowing a fluid through the original physical three-dimensional model. The method also includes measuring a first sound generated of the fluid through the original physical three-dimensional model. The flowing the fluid through a modified physical three-dimensional model devoid of the malformation and measuring a second sound generated of the fluid through the modified physical three-dimensional model. The method also includes comparing the first and second sounds to determine the efficacy of removal of the malformation/abnormality.

According to one aspect of the above embodiment, the one or more images may be obtained using one of X-ray computed tomography, computerized axial tomography scanning, or magnetic resonance imaging.

According to one aspect of the above embodiment, the physical three-dimensional model may be formed using additive manufacturing.

### BRIEF DESCRIPTION OF THE DRAWINGS

Embodiments of the present disclosure are described herein with reference to the accompanying drawings, wherein:

FIG. 1 is a two-dimensional cross-sectional velocimetry image of blood flow through a sigmoid sinus and a jugular vein;

FIG. 2 is a side, partially cross-sectional view of a catheter having a transducer disposed at a distal portion thereof according to one embodiment of the present disclosure;

FIG. 3A is a side, partially cross-sectional view of a catheter having a plurality of transducers disposed in a linear array at a distal portion thereof according to another embodiment of the present disclosure;

FIG. 3B is a side, partially cross-sectional view of a catheter having a plurality of transducers disposed in a spiral configuration at a distal portion thereof according to another embodiment of the present disclosure;

FIG. 4 is a schematic diagram of a system including a catheter with a transducer and a driver circuit for transducing sound within a blood vessel according to the present disclosure;

FIG. 5 is a flow chart of a method for manufacturing a three-dimensional printed model of transverse sinus anatomy and procedure planning according to the present disclosure;

FIG. 6A is a photograph of a cross-section of a three-dimensional printed model of transverse sinus anatomy of a patient suffering from pulsatile tinnitus before a lumbar puncture procedure;

FIG. 6B is a photograph of a cross-section of a three-dimensional printed model of transverse sinus anatomy of the patient suffering from pulsatile tinnitus after the lumbar puncture procedure;

FIG. 7A is a plot of amplitude of the sound recorded by an electronic stethoscope disposed on an outer surface of the pre-lumbar puncture pulsatile model of FIG. 6A;

FIG. 7B is a plot of amplitude of the sound recorded by the catheter of FIG. 2 disposed inside the transverse sinus anatomy of the pre-lumbar puncture pulsatile model of FIG. 6A;

FIG. 8A is a plot of amplitude of the sound recorded by the electronic stethoscope disposed on the outer surface of the post-lumbar puncture pulsatile model of FIG. 6B; and

FIG. 8B is a plot of amplitude of the sound recorded by the catheter of FIG. 2 disposed inside the transverse sinus anatomy of the post-lumbar puncture pulsatile model of FIG. 6B.

### DETAILED DESCRIPTION

Embodiments of the present disclosure are described in detail with reference to the drawings, in which like reference numerals designate identical or corresponding elements in each of the several views. As used herein the term “proximal” refers to the portion of a device that is closer to the user, while the term “distal” refers to the portion that is farther from the user.

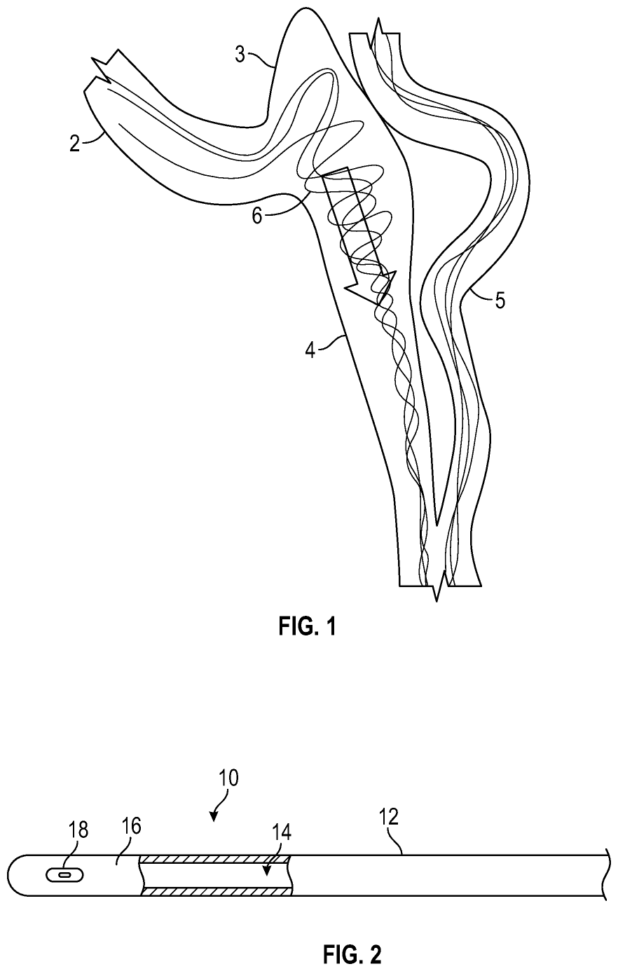

FIG. 1 shows a velocimetry image of irregular blood flow through SSIJ of a patient suffering from pulsatile tinnitus. In particular, FIG. 1 shows a sigmoid sinus 2, a jugular bulb 3, a jugular vein 4, and a carotid artery 5, and blood flow 6 through the jugular vein 4. More specifically, blood flow 6 includes a vortex flow pattern originating in a superior aspect of the jugular bulb 3 and propagating down the descending jugular vein 4. It is believed that the blood flow 6, and in particular, its vortical shape, is a source of sound generation that is picked up by the cochlea and experienced by the patient as pulsatile tinnitus.

With reference to FIG. 2, a catheter 10 includes an elongated shaft 12 defining an elongated inner lumen 14. The elongated shaft 12 may have an outer diameter from about 0.3 mm to about 2 mm, in embodiments from about 0.5 mm to about 1 mm, such that the catheter 10 is configured to pass into the jugular vein 4, the carotid artery 5, or other blood vessels near the cochlea. The catheter 10 includes a distal portion 16 and the catheter 10 may be of any suitable length for reaching the desired anatomical area. The elongated shaft 12 may be formed from a resilient flexible material, which may be a biocompatible polymer such as polyurethane. The elongated shaft 12 may be coupled to a hub (not shown) or any other structural elements that are disposed outside a patient during use to facilitate the use of the catheter 10.

In embodiments, the elongated shaft 12 may be formed as a single layer sheath, or a multi-layer sheath. The multi-layer sheath may be a laminated sheath in which each layer is formed from same or different materials. In embodiments, the elongated shaft 12 may be braided. In further embodiments, the elongated shaft 12 may include various coatings on an inner surface of the shaft 12, i.e., within the lumen 14, or on an outer surface of the shaft 12.

The catheter 10 includes a transducer 18, which may be disposed on the outer surface of the elongated shaft 12 or within the lumen 14. In embodiments, the transducer 18 may be coupled at a distal end 17A of the distal portion 16. The transducer 18 may be coupled to the elongated shaft 12 using waterproof adhesive and/or the elongated shaft 12 may be laminated by a film to secure the transducer 18 to the elongated shaft 12.

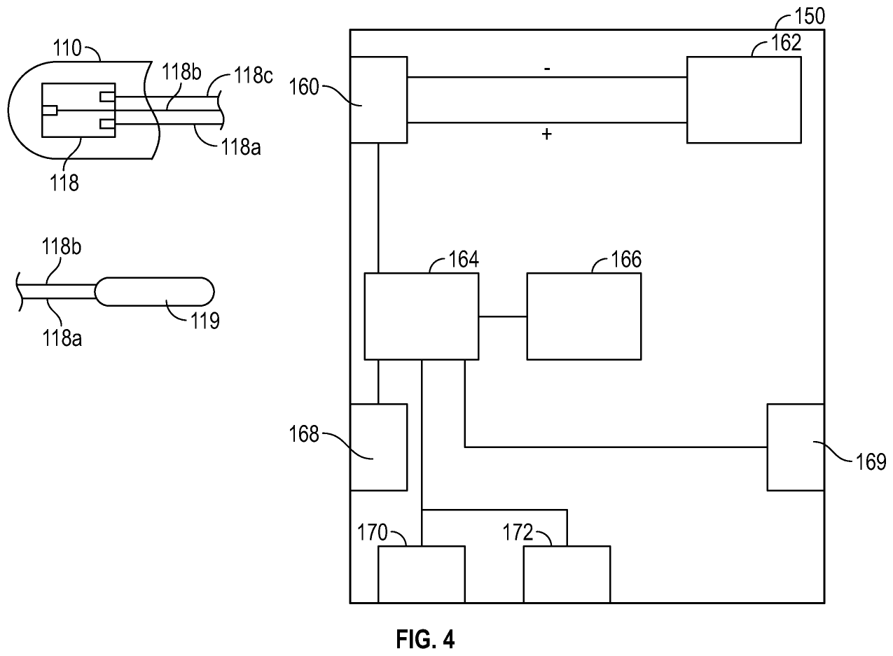

The transducer 18 may be any transducer that can be fitted on the elongated shaft 12 without significantly increasing the diameter of the elongated shaft 12. The transducer 18 may be a micro-electromechanical systems (MEMS) microphone or an electret diaphragm microphone, or any other millimeter scale microphone. In further embodiments, the transducer 18 may be a flexible membrane transducer. The transducer 18 may be coupled to an electrical lead which couples the transducer 18 to a driver circuit, such as, an interface device 150 (FIG. 4.)

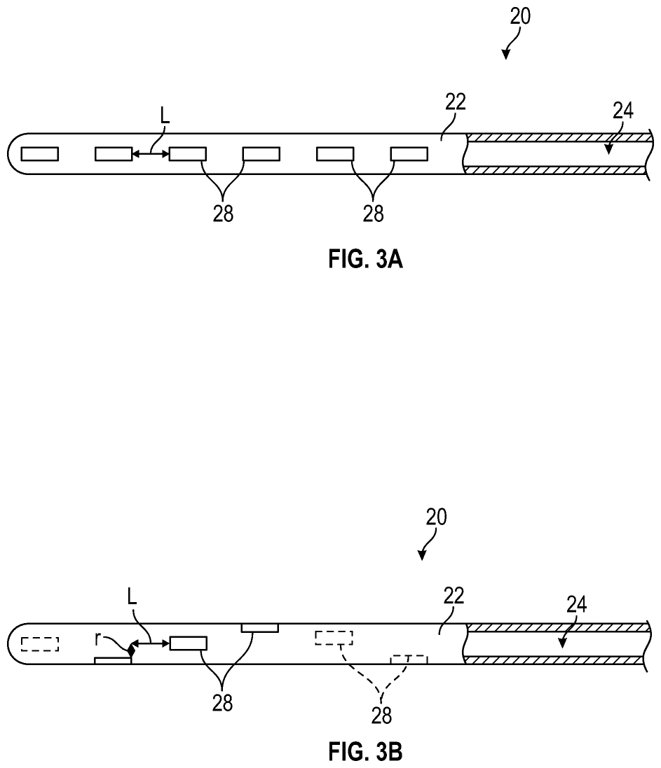

With reference to FIGS. 3A and 3B, another embodiment of a catheter 20 is shown. The catheter 20 is substantially similar to the catheter 10 and includes an elongated shaft 22 defining a lumen 24. The catheter 20 has the same dimensions as the catheter 10 and may be formed from the same materials. The catheter 20 includes a plurality of transducers 28 disposed at a distal portion 26 of the elongated shaft 22. The transducers 28 are substantially similar to the transducer 18 and may be arranged in an array. As shown in FIG. 3A, the transducers 28 may be arranged in a linear array with each of the transducers 28 being separated by a predetermined longitudinal distance, l. In further embodiments, as shown in FIG. 3B, the transducers 28 may be arranged in a spiral array with each of the transducers 28 being separated by a predetermined longitudinal distance, l, and a radial distance, r.

With reference to FIG. 4, a system 100 including a catheter 110, which may be either the catheter 10 of FIG. 2 or the catheter 20 of FIG. 3. The catheter 110 includes a transducer 118, which is representative of the transducer 18 or 28, which is shown schematically as having three leads 118a, 118b, 118c for connecting to ground, power, and output, respectively. The three leads 118a, 118b, 118c are coupled to an interface device 150, which includes a catheter connector 160, a power section 162, and a controller 164 coupled to a memory device 166.

The controller 164 may be any standard processor capable of executing data structures, data sequences, and/or computer programs. The memory device 166 may be any memory component associated with the controller 164 for storing information as data structures, data sequences, and/or computer programs. The data structures, data sequences, and/or computer programs may be sequences of data structures or computer executable program code, and may be routines or subroutines within the structures or code.

The controller 164 may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

The controller 164 may be operably connected to the memory device 166, which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The controller 164 and the memory device 166 may be any standard processor and memory component known in the art.

The interface device 150 may also include a display 168, one or more connectors 170 for coupling additional computing devices, and an audio output 172 for outputting the sound picked up by the transducer 118. The audio output 172 may be a speaker or an audio connector configured to couple to an audio playback device, e.g., headphones or speaker. The interface device 150 also includes a user interface 169, which may include one or more buttons, a touch screen, a keyboard, and the like, allowing for a user to control the interface device 150. The user interface 169 may be configured to activate or deactivate the transducer 118 as well as commence or stop sound recording and/or playback.

Sound transducers such as the transducer 118 utilize a driver circuit in order to function, namely, output an electrical signal in response to an external physical stimulus. The transducer 118 may have an internal or an external power source. In embodiments, the transducer 118 may have a power source (e.g. a battery 119) disposed at the distal portion 16 or 26 of the catheter 10 or 20. In embodiments, the power section 162, which may be an AC-DC power supply, supplies electrical energy to the transducer 118 and components of the interface device 150. The power section 162 is coupled to the transducer 118 through the leads 118a and 118b, which are disposed within the lumen 14 or 24 of the catheter 10 or 20, respectively. The leads 118a and 118b terminate at the catheter connector 160.

In further embodiments, the transducer 118 may be coupled to the interface device 150 wirelessly, thus obviating the need to use electrical leads to couple the transducer 118 to the interface device 150. Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 802.15.4-2003 standard for wireless personal area networks (WPANs)).

The transducer 118 is configured to output a digital signal corresponding to the sound as a signal through the lead 118c, which also terminates at the catheter connector 160. The digital signal is transmitted through the lead 118c to the controller 164, which receives and processes the digital signal and outputs the same or a processed audio signal through the audio output 172. The controller 164 is also configured to store the digital signal within the memory device 166 and is further configured to transmit the signal for further processing and/or storage to an external computing device. The sound may be output through the audio output 172 simultaneously while the digital signal is being stored.

During use, the catheter 10 is inserted in the arterial or venous system using conventional techniques. The insertion point may be anywhere in the body. In embodiments, a transvenous via a femoral or jugular approach may be used. The catheter 10 may be advanced into, or near a suspected source of sound/turbulence generation and the recording and playback started. In embodiments, the catheter 10 may be advanced into a blood vessel near the cochlea, such as the jugular vein 4 and the carotid artery 5 (FIG. 1). The sound picked up by the transducer 18 of the catheter 10 may be played into the room such that the patient can hear the sound, which would facilitate the patient being able to verify that the sound is the same as the sound the patient is experiencing as pulsatile tinnitus. The catheter 10 may then be moved away from the sound generating portion of the blood vessel and the precipitous decline in amplitude of the sound is used as a confirmation of the location of sound production.

Detection of largest amplitude and the decline associated with movement of the catheter 10 may be determined by the controller 164. In particular, the controller 164 continuously receives the electrical signal from the transducer 118 and determines the amplitude of the electrical signal. The controller 164 also stores the amplitude, at least temporarily, and compares the most recent amplitude to previously stored amplitude at a prior time period, which may be done at a frequency from about 100 milliseconds to about 1 second. In addition, the controller 164 may analyze a subset of stored amplitudes to determine if there is a continual decline. Thus, if there is a plurality samples of continuously decreasing amplitude, then the controller 164 determines that the location of sound was identified. The location of the sound may be determined by correlating the instance at which the largest amplitude was recorded with the insertion distance of the catheter 10.

In cases when the sound source is unknown, the catheter 10 may also be used while being withdrawn. Initially, the catheter 10 is positioned at a distal most portion of a blood vessel and the catheter 10 is then moved in a proximal direction, namely, toward the insertion site. At regular intervals during the withdrawal of the catheter 19, sound is recorded, measured, and analyzed by the interface device 150. The location at which the transducer 18 recorded a sound having the largest amplitude is the closest to the sound source and may be determined by the controller 164 as described above.

In further embodiments, depending on the size of the catheter 10, the catheter 10 may have flow disruption properties and may transiently cease sound transmission. Removing the catheter 10 entirely from this area may result in return of measurable/audible sound. This technique may also be used to identify the source of sound generation.

The catheter 20 may be used in a similar manner as the catheter 10, in particular, the catheter 20 may be used to measure sound while being advanced and/or withdrawn through the blood vessel while measuring sounds. The array of transducers 28 may measure sound simultaneously, or sequentially, or in groups. Information from the multiple transducers 28 is used to triangulate the source of sound. In particular, the amplitude of the sound measured at each of the transducers 28 is different since each of the transducers 28 is disposed at a specific distance from the sound source. This would obviate the need for the catheter 20 to be positioned immediately near the source of sound production in order to define the location of the source. Furthermore, since multi-array design uses triangulation to identify the location of sound/turbulence, this would also allow for accurate diagnosis of disease without requiring the catheter 20 to be positioned in multiple locations. A single location of insertion would allow the catheter 20 to triangulate disease throughout the body. The triangulation is performed automatically by the controller 164, which compares the amplitude of the sound picked up by each of the transducers 28.

The interface device 150 is also configured to record the signals from the transducers 18 and 28 of the catheters 10 and 20, respectively. The memory device 166 stores each of the recorded signals in a database as a sound signature entry. Each entry corresponds to the recorded sound and includes an audio file as well as the underlying cause for the sound. Such causes include, but are not limited to, atherosclerotic disease in an artery, venous sinus stenosis, dural arteriovenous fistula, and combinations thereof. In addition, each entry also includes other properties associates with the sound, including but not limited to, amplitude, frequency, whether the sound is rhythmic or arrhythmic, whether the sound pulse is synchronous, duration of the sound, whether the sound is intermittent, whether the sound changes in response to Valsalva maneuver or other patient maneuvers, and the location of sound, maximum amplitude of the sound.

Sound signatures generated in a larger vessel with slower blood flow velocity, such as a dural venous sinus, are often lower pitched and lower in amplitude than sounds generated by smaller vessels that have high blood flow velocity such as the arteries. Sounds recorded by the catheters 10 and 20 are different than those heard outside the patient, e.g., by using a stethoscope due to the sound attenuating properties of the soft tissues overlying the blood vessels, including in some cases the skull. The unique ability to position the catheters 10 and 20 within the vessels allows for a more accurate recording and a more accurate diagnosis than with conventional devices.

The database of sound signatures that are recorded by the interface device 150 and the associated diagnoses and other properties allow the interface device 150 to automatically diagnose future patients. It is envisioned that there will be an ongoing training of the device using artificial intelligence after the initial database is developed until diagnostic accuracy is achieved.

The terms “artificial intelligence,” “data models,” or “machine learning” may include, but are not limited to, neural networks, convolutional neural networks (CNN), recurrent neural networks (RNN), generative adversarial networks (GAN), Bayesian Regression, Naive Bayes, nearest neighbors, least squares, means, and support vector regression, among other data science and artificial science techniques.

A neural network may be used to train a diagnosing tool. In various embodiments, the neural network may include a temporal convolutional network, with one or more fully connected layers, or a feed forward network. In various embodiments, training of the neural network may happen on a separate system, e.g., graphic processor unit (“GPU”) workstations, high performing computer clusters, etc., and the trained diagnosing tool would then be deployed on the interface device 150. In further embodiments, training of the neural networks may happen locally, e.g., on the interface device 150. After training, the diagnosing tool may be a software application that is stored in the memory device 166 and is executable by the controller 164 to diagnose the recorded sound.

The catheters and system according to the present disclosure may also be used to diagnose a variety of other blood vessel abnormalities that act as sound generation sources. Vascular turbulence is often involved in pathogenesis of cerebrovascular diseases such as, atherosclerosis, dissections, aneurysm development, dural arteriovenous fistula, arteriovenous malformations, and the like.

The catheters and the system according to the present disclosure would facilitate early and accurate diagnosis by precisely localizing a source of sound generation due to vascular turbulence. In particular, identifying turbulent flow in the blood vessels may be used to predict development of disease and/or identify progression of a disease prior to anatomic changes by identifying the flow perturbation, which may lead to earlier and safer therapies. This may be done by comparing the recorded sound to sound signatures stored in a database as described above.

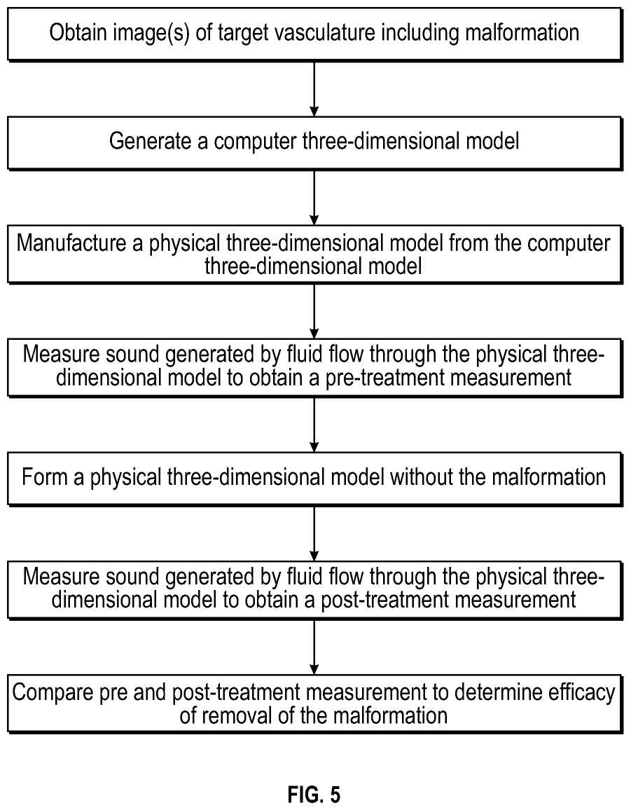

With reference to FIG. 5, a method for manufacturing a three-dimensional printed model of transverse sinus anatomy and treatment planning initially includes obtaining an image of the target vasculature or any other segment that is being treated. Thus, for treatment and diagnosis of pulsatile tinnitus SSIJ is imaged. Images may be obtained using any suitable medical imaging technology, such as, X-ray computed tomography, computerized axial tomography scanning, magnetic resonance imaging, and the like. Once images are obtained, the images are used to generate a computer three-dimensional model. The computer three-dimensional model may be generated at a computer running software that receives as input one or more images of the target vasculature and outputs the computer three-dimensional model. The computer three-dimensional model may be generated by segmenting the images to obtain contours and/or surfaces of the target vasculature. Thereafter, the computer three-dimensional model may be further processed to generate a mesh model. In addition, the model may be automatically or manually adjusted to modify certain geometries of the three-dimensional model. Once the three-dimensional model is finalized, a stereolithography (“STL”) file may be generated that is supplied to a three-dimensional printer, which may use the three-dimensional model to form a physical three-dimensional model by additive manufacturing (e.g., three-dimensional printer) or any other suitable manufacturing technique.

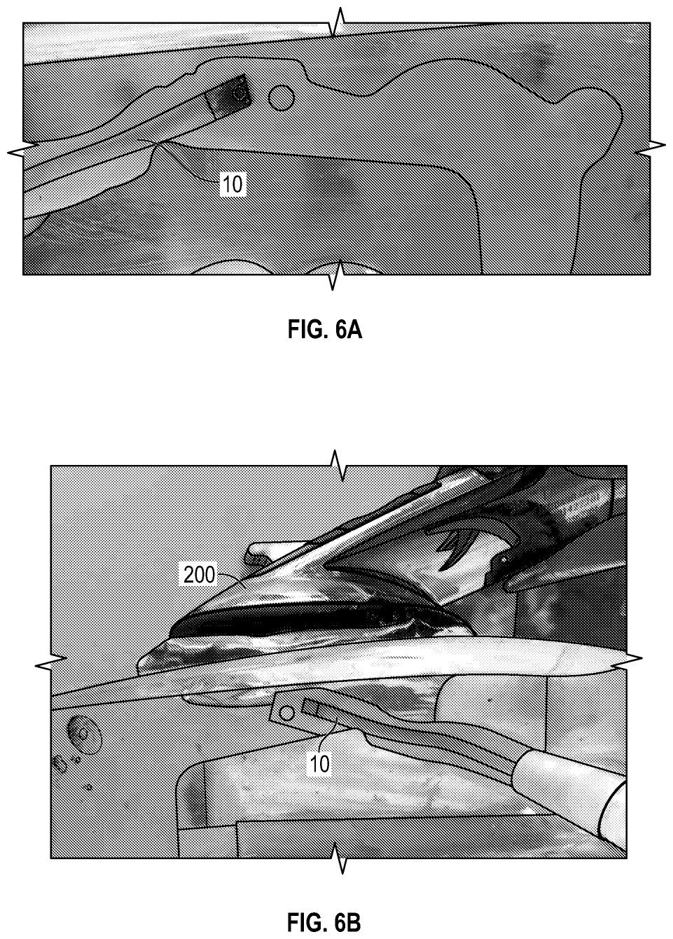

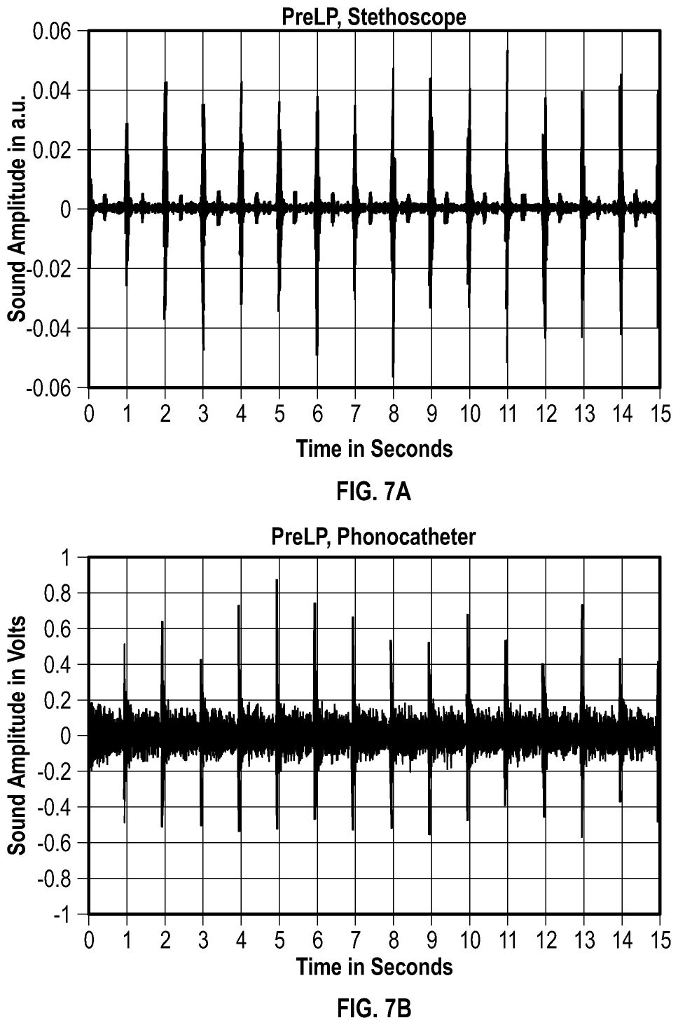

The target vasculature may include a malformation that is represented in the computer and physical three-dimensional models. The malformation, e.g., stenosis, may be responsible for the blood flow pattern causing pulsatile tinnitus. The presence of the malformation is used for procedure planning and testing the proposed treatment, e.g., lumbar puncture. In particular, the three-dimensional model may be coupled to a fluid loop simulating blood flow. In embodiments, a pump may be used to circulate a glycerol-water mixture or any other suitable fluid to mimic blood flow. The catheter 10 and/or an external microphone, e.g., an electronic stethoscope 200, as shown in FIGS. 6A and 6B, may be used to record transluminal and intravascular sounds, respectively, generated due to the presence of the malformation. The recorded sounds may be used to obtain a pre-treatment, e.g., baseline, measurements as shown in FIGS. 7A and 7B and plotted as sound amplitude as a function of time plots. The pre-treatment measurements are used to determine the efficacy of the proposed treatment.

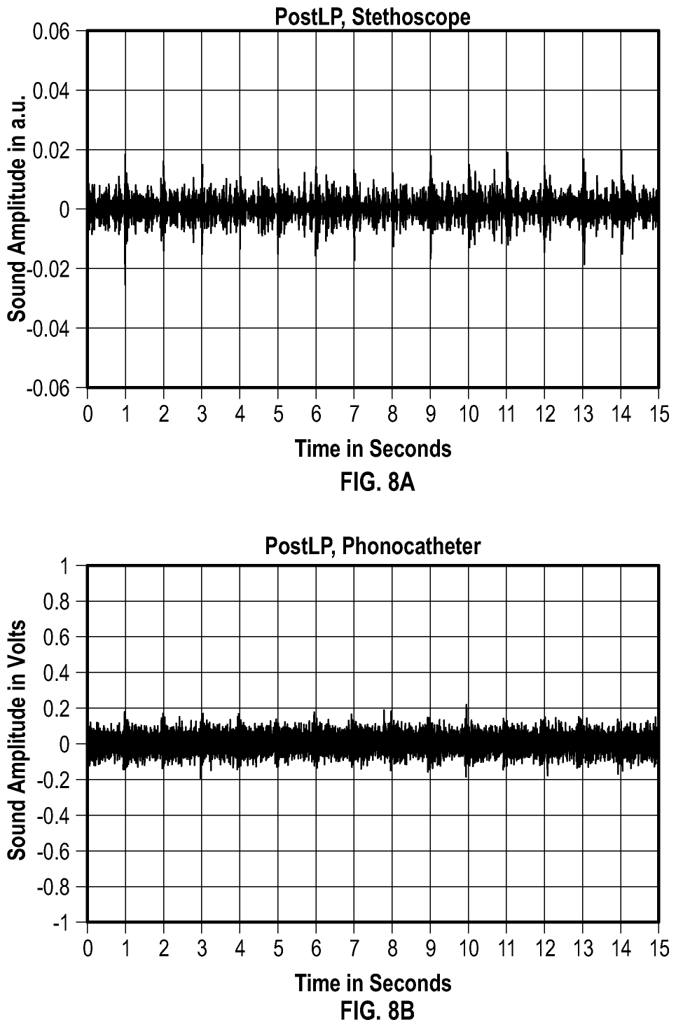

Thereafter, another three-dimensional model may be printed by generating a new computer three-dimensional model without the malformation. In embodiments, the original physical three-dimensional model may be modified to remove the malformation. The modification may include a proposed treatment to remove the malformation. Thereafter, the modified three-dimensional model may then be subjected to the same flow testing and sound measurements described above with respect to the original physical three-dimensional model to obtain post-treatment sound plots as shown in FIGS. 8A and 8B. The plots of FIGS. 8A and 8B are then compared with the plots of FIGS. 7A and 7B to determine the efficacy of the proposed treatment in removing the pulsatile sound responsible for pulsatile tinnitus. Thus, if the sound plots of the modified three-dimensional model illustrate that the amplitude of the pulses has decreased, then the proposed treatment procedure is deemed to be successful and may be implemented on the patient.

The following Example illustrates an embodiment of the present disclosure. This Example is intended to be illustrative only and is not intended to limit the scope of the present disclosure.

### EXAMPLE

This example describes using a catheter according to the present disclosure to record sound.

With reference to FIGS. 6A and 6B, two benchtop three-dimensional printed pulsatile tinnitus models (pre-LP and post-LP models) that represent a pulsatile tinnitus patient's transverse sinus anatomy before and after lumbar puncture (LP) were used to determine the ability of the catheter 10 to measure changes in sound generation. The patient whose sinus anatomy was modeled reported a resolution of her PT symptoms after the lumbar puncture. The post-LP model used in this Example reflects the anatomical change in the transverse sinus. There was also a reduction in stenosis due to the LP.

A glycerol-water mixture was pumped through each of the models to mimic blood flow at a mean flow rate of about 7.4 cc/s. An external pump (not shown) simulated a cardiac rhythm (about 60 beats per minute) was connected to the flow models to circulate the glycerol-water mixture at the characteristic flow rate of about 7.4 cc/s. The catheter 10 was inserted through a 9 French size (Fr) access port and was navigated into a stenosis region (FIGS. 6A and 6B). The stethoscope 200 was placed externally over the same stenosis region to record pulsatile tinnitus transluminally and validate the sound measurements recorded by catheter 10. Ultrasound transmission gel was used to couple the stethoscope 200 to the surface of the model.

Both the electronic stethoscope 200 and the catheter 10 were able to record transluminal and intravascular sounds generated from the stenosis. Variation in peak to root mean square (RMS) sound amplitude values from the transverse sinus in pre-LP and post-LP models was calculated for the sound measurements obtained by both the catheter 10 and the electronic stethoscope 200. Wilcoxon rank sum test was also used to statistically determine the differences in measurements between the patient-specific models.

Sounds recorded from the model representing the transverse sinus anatomy with stenosis before lumbar puncture with the stethoscope 200 (FIG. 7A) and catheter 10 (FIG. 7B) suggest that the sound was synchronous with the cardiac rhythm mimicking pulsatile tinnitus. Sounds recorded from the model representing the transverse sinus anatomy immediately after lumbar puncture with the stethoscope 200 (FIG. 8A) and the catheter 10 (FIG. 8B) demonstrate that the sound amplitude was weaker compared to the pre-LP model.

The catheter 10 was in good agreement with the electronic stethoscope 200 demonstrating that the peak-to-rms (mean±standard deviation) sound amplitude was significantly louder (p<0.0001) in the stenosis region in pre-LP model (Stethoscope: 9.03±1.61; Phonocatheter: 6.62±1.55) as shown in FIGS. 7A and 7B as compared to the same region in post-LP model (Stethoscope: 4.20±0.86; Phonocatheter: 3.62±0.88) (FIGS. 8A and 8B).

It will be understood that various modifications may be made to the embodiments disclosed herein. In particular, the catheter according to the present disclosure may be used in any suitable blood vessel. Therefore, the above description should not be construed as limiting, but merely as exemplifications of various embodiments. Those skilled in the art will envision other modifications within the scope and spirit of the claims appended thereto.

It will be appreciated that of the above-disclosed and other features and functions, or alternatives thereof, may be desirably combined into many other different systems or applications. Various presently unforeseen or unanticipated alternatives, modifications, variations or improvements may be subsequently made by those skilled in the art which are also intended to be encompassed by the following claims. Unless specifically recited in a claim, steps or components of claims should not be implied or imported from the specification or any other claims as to any particular order, number, position, size, shape, angle, or material.

What is claimed is:

1. A catheter for diagnosing an abnormality of a blood vessel, the catheter comprising:
an elongated body defining an elongated lumen therethrough; and
at least one transducer disposed on an outer surface of the elongated body, the at least one transducer configured to output an electrical signal in response to sound.

2. The catheter according to claim 1, wherein the elongated body has an outer diameter from about 0.5 mm to about 1.0 mm.

3. The catheter according to claim 1, wherein the at least one transducer includes a plurality of transducers.

4. The catheter according to claim 3, wherein the plurality of transducers is arranged in an array.

5. The catheter according to claim 4, wherein the array is one of a linear array or a spiral array.

6. The catheter according to claim 1, further comprising:
a battery disposed within the elongated lumen, the battery coupled to the at least one transducer.

7. A system for diagnosing an abnormality of a blood vessel, the system comprising:
a catheter including:
an elongated body defining an elongated lumen therethrough; and
at least one transducer disposed on an outer surface of the elongated body, the at least one transducer configured to output an electrical signal in response to sound; and
interface device coupled to the at least one transducer, the interface device including a controller configured to process the electrical signal and to record the sound.

8. The system according to claim 7, wherein the elongated body has an outer diameter from about 0.5 mm to about 1.0 mm.

9. The system according to claim 7, wherein the at least one transducer includes a plurality of transducers.

10. The system according to claim 9, wherein the plurality of transducers is arranged in one of a linear array or a spiral array.

11. The system according to claim 7, further comprising:
a battery disposed within the elongated lumen, the battery coupled to the at least one transducer.

12. The system according to claim 7, wherein the controller is further configured to output the electrical signal through an audio output.

13. The system according to claim 7, wherein the interface device further includes a memory device configured to store a record of the sound.

14. The system according to claim 13, wherein the memory device is configured to store a database of a plurality of entries, each of which pertains to a record of the sound.

15. The system according to claim 14, wherein each entry of the plurality of entries includes at least one property describing the sound, the at least one property selected from the group consisting of amplitude, frequency, rhythm, and synchronicity.

16. The system according to claim 14, wherein the memory device stores a diagnosing tool executable by the controller, the diagnosing tool configured to automatically diagnose an abnormality of a blood vessel based on the plurality of entries of the database.

17. A method for diagnosing an abnormality of a blood vessel, the method comprising:
placing a catheter into a blood vessel near a cochlea, the catheter including an elongated body defining an elongated lumen therethrough;
measuring a sound at a transducer disposed on an outer surface of the elongated body, the transducer configured to output an electrical signal in response to sound; and
processing the electrical signal at a controller to determine location of the sound.

18. The method according to claim 17, wherein the sound is measured while the catheter is being withdrawn from the blood vessel.

19. The method according to claim 17, further comprising:
comparing the sound at the controller to a database of a plurality of sounds and corresponding parameters to automatically diagnose the abnormality, wherein the abnormality is at least one of an atherosclerosis, a dissection, an aneurysm, a dural arteriovenous fistula, an arteriovenous malformation, or pulsatile tinnitus.
